(19) United States
(12) Patent Application Publication
(10) Pub. No.: US 2009/0069627 A1
(43) Pub. Date: Mar. 12, 2009

(54) Device for supporting the abdominal wall relative to underlying organs during minimally invasive surgery

(76) Inventor: Hans Haindl, Wennigsen (DE)

Correspondence Address: SHLESINGER, ARKWRIGHT & GARVEY LLP, 1420 KING STREET, SUITE 600, ALEXANDRIA, VA, 22314, US

(21) Appl. No.: 12/153,010

(22) Filed: May 12, 2008

Related U.S. Application Data
(63) Continuation of application No. PCT/EP2006/009216, filed on Sep. 22, 2006.

(30) Foreign Application Priority Data
Nov. 11, 2005 (DE) 10 2005 053 831.2

(51) Int. Cl. A61B 1/32

(52) U.S. Cl. 600/37; 600/201

(57) ABSTRACT

Device for supporting the abdominal wall away from organs positioned thereunder for freeing up a surgical space in minimally invasive surgery, having multiple tube-shaped hollow bodies, which are fillable via a hose line (6, 16), and which are connected to each other in circumferential direction to form a preferably conical toroidal shape. The elongated hollow bodies (2, 10) are each supported at their ends (4, 12) on a tube-shaped, thin-walled, foldable hollow body (8, 14), which forms a ring, and which can be filled via a hose line (6, 16), extending along the two edges of the toroidal shape. The diameters of the two annular hollow bodies (8, 14) may be the same, or different, thus forming a cylindrical or frustoconical wall (18) serving for support.

### FIELD OF THE INVENTION

The invention relates to a device for supporting the abdominal wall away from organs positioned thereunder for freeing up a surgical space in minimally invasive surgery.

### BACKGROUND OF THE INVENTION

With minimally invasive surgery of the abdominal cavity it is generally known to fill the same with carbon dioxide gas in order to thereby form a gas-filled hollow space, in which sufficient room exists for the surgeon to work, using instruments inserted through the abdominal wall. This generally known method has multiple disadvantages. One disadvantage is that the pressure in the abdominal cavity causes a degradation of the venous return, therefore leading to hemodynamical effects that may have an adverse effect on the patient. Another disadvantage is that the carbon dioxide is resorbed through the abdominal membrane to a significant extent, which causes a shifting of the metabolic status of the patient, leading up to acidemia. This carbon dioxide must be exhaled during the anesthesia; however, acidemia of the tissue still cannot be avoided. Acidemia of the tissue also does not aid in the healing of a wound.

A further disadvantage is that although the filled-in carbon dioxide does create a clearly visible gas bubble within the abdominal space, the same is not always positioned at the correct location, thus resulting in the fact that the surgery site to which the surgeon wishes to gain access is not accessible, and may even be obstructed by other structures, such as intestinal loops. Since these intestinal loops must be held aside using correspondingly small instruments inserted through the abdominal cavity, the surgery requires additional staff, only to hold organs aside. Since organs that are held aside, such as intestinal loops, are extremely sensitive, the risk of injuries during the holding aside using metallic instruments, such as finger retractors, exists.

In order to avoid these disadvantages, various devices are known, by means of which the abdominal cavity may be raised, or supported, respectively. A device is known from CA 2 109 795, in which radially extending arms are arranged on the ends of rods that are retained parallel to each other in a pivoting manner. These arms are positioned directly next to each other in the idle position so that they can be inserted into an opening in the abdominal wall. These two arms are spread apart inside of the abdominal cavity in the manner of a fan by means of pivoting so that they engage beneath the abdominal wall, which can thus be raised by means of the device. A similar device is known from U.S. Pat. No. 5,820,555. One disadvantage of both known devices is that the devices must be continuously held in the raised position. This is carried out in the case of the above-named US patent specification by means of a support arranged laterally of the patient.

A device is known from U.S. Pat. No. 5,613,939, which functions in the same manner as the previously described devices, and which differs from the same in that the arms are formed by flexible, elongated hollow bodies, which are initially folded together during the inserting through an opening in the abdominal wall, and which are then pneumatically or hydraulically filled inside of the abdominal cavity so that they are rigid, and engage beneath the abdominal wall. The disadvantages stated for the previously described known devices also apply to this known device in the same manner.

A device of the type stated in the preamble of patent claim 1 for raising, or supporting, the abdominal wall away from organs positioned beneath for the purpose of minimally invasive surgery is known from U.S. Pat. No. 5,520,609, which is comprised of multiple thin-walled, foldable, tube-shaped hollow bodies forming a cylindrical shape, which can be filled via a hose line. For this purpose, the hollow bodies have the shape of rings, which are connected to each other in axial direction, and which are each connected to a hose line, via which they may be inflated after the insertion into an abdominal cavity. This known device is relatively complicated, has a low lateral rigidity, and requires a relatively large wall thickness, which complicates the insertion through a small opening in the abdominal wall.

### OBJECTS AND SUMMARY OF THE INVENTION

The invention is based on the object of creating a device of the type stated in the preamble of claim 1, which is easy to produce, and which can be inserted into the abdominal cavity through a relatively small opening in the abdominal wall.

The object of the invention is achieved by the teachings set forth in the characteristics of claim 1.

The fundamental idea of the invention is to form the tube-shaped hollow bodies in an elongated manner so that they form rigid rods after filling the same with a gas, or with a fluid, which are each supported at their ends on a tube-shaped hollow body, which forms a ring, and which can be filled via a hose line, such that the elongated hollow bodies form a truncated wall, which is inserted into the abdominal cavity through a small opening in the abdominal wall in their folded position, and there are brought into a rigid toroidal shape by means of filling the hollow bodies so that they retain the abdominal wall at a distance to the surgery site. The toroidal shape can be cylindrical or truncated, wherein it is purposeful to position the edge of the truncated shape having the smaller diameter onto the surgery region. The device is therefore capable of sufficiently supporting the abdominal wall, and to free up the surgery region from laterally invading organ structures, such as intestinal loops. The interior spaces of multiple, or of all hollow bodies can be connected to each other so that only one hose line is, or only few hose lines are necessary for the filling thereof.

According to a further embodiment of the invention the interior spaces of the elongated hollow bodies are connected in an alternating manner in axial direction to the hollow space of the one hollow space, and to the hollow space of the other hollow body forming a ring, wherein the annular hollow bodies each have dedicated hose lines for filling their hollow spaces, and for filling the hollow spaces of the elongated bodies connected to the same. Thus, two support systems are formed, which are independent of each other so that with a damage of one of the hollow bodies, such as by means of a surgical instrument, sufficient support of the abdominal wall is still ensured.

According to a further embodiment of the invention the hollow bodies are formed by means of two plastic foils, which are fused together in the area of the limitations of the hollow bodies. These plastic foils can be very thin so that they can be inserted in their folded position into the abdominal cavity through a relatively small opening in the abdominal wall, subsequent to which they can be transformed into rigid bodies by means of pneumatic or hydraulic filling, which are capable of raising and supporting the abdominal wall, thus keeping the surgery site free.

Purposefully, protrusions are mounted on one, or both of the hollow bodies, each forming a ring, wherein the protrusions can be connected to adjacent body parts, particularly to the abdominal wall by means of a surgical thread, in order to secure the device in this manner. These protrusions can have the shape of individual tabs, or circumferential protrusions, such as protruding welded edges.

The invention will be explained in further detail based on an exemplary embodiment by means of the drawings.

### BRIEF DESCRIPTION OF THE DRAWINGS

FIG. 1 illustrates the embodiment according to the invention in a side view, and

FIG. 2 illustrates the embodiment according to the invention of FIG. 1 in a perspective view.

### DETAILED DESCRIPTION OF THE INVENTION

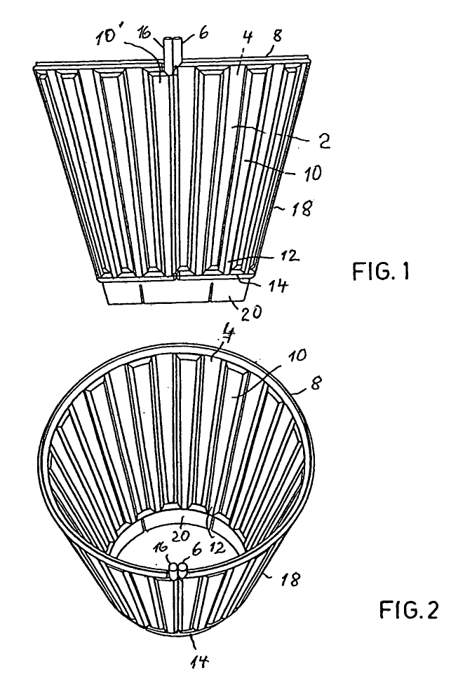

The device according to FIG. 1 has elongated, thin-walled and foldable hollow bodies 2, which are each supported at their upper ends on a tube-shaped, thin-walled hollow body 8, which forms a ring, and which can be filled via a hose line 6, wherein the interior of the tube-shaped hollow body 2 and of the tube-shaped hollow body 8 are connected to each other so that both hollow bodies can be filled with gas or a fluid via the hose line 6.

The device further has thin-walled and foldable hollow bodies 10 embodied in the same manner as the hollow bodies 2, which are supported at their lower ends 12 on a thin-walled, foldable tube-shaped hollow body 14, which forms a ring, wherein the interior of the tube-shaped body 10 is connected to each interior of the annular, tube-shaped hollow body 14. The interior of one of the tube-shaped bodies 10, labeled as 10′ in the drawing, is connected to a hose line 16 so that the interior of the tube-shaped body 10′, and also the interior of the annular, tube-shaped body 14 can be filled with gas or a fluid, and thus also the interior of all tube-shaped hollow bodies 10 connected thereto can be filled.

All elongated, tube-shaped hollow bodies 2 and 10 are connected to each other laterally relative to their direction of elongation. The annular hollow body 8 has a larger diameter than the annular hollow body 14 so that hollow bodies 2 and 10 connected to each other form a frustoconical wall 18, which can be displaced into a rigid position, if all hollow bodies 2, 10, 8, and 14 are tightly filled by means of introducing, for example, a fluid into the hose lines 6. This means that in this position, for example, the annular hollow body 8 holds an abdominal wall at a distance from the interior body part, against which the annular hollow body 14 abuts.

Tabs 20 are positioned on the lower annular hollow body 14, by which the entire device can be secured by one or multiple surgical threads.

FIG. 2 shows the embodiment of FIG. 1 in a perspective illustration so that it is readily understood that the wall 18 as a whole forms a conical hollow cylinder.

The interiors of the elongated hollow bodies 2, and the interior of the annular hollow body 8, and also the interiors of the elongated hollow bodies 10 and of the annular hollow body 14 form two separate systems, which can each be filled separately via the hose lines 6 or 15. This means that in case of damage to one hollow body of one of the two groups, the hollow bodies of the other group may continue to maintain their supporting action. This results in an increased reliability during surgery.

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention or limits of the claims appended hereto.

1-8. (canceled)

9. A device for supporting the abdominal wall away from organs positioned thereunder for freeing up a surgical space in minimally invasive surgery, comprising:
a) multiple tube-shaped, thin-walled, foldable hollow bodies forming a toroidal shape, and being fillable via a hose line;
b) the tube-shaped hollow bodies being elongated, and extending in the direction of the toroidal shape;
c) the tube-shaped hollow bodies being connected to each other in a circumferential direction of the toroidal shape; and
d) the elongated bodies being supported in an axial direction of the toroidal shape at their ends on a respective tube-shaped, thin-walled, foldable hollow body, which form a ring, and being fillable via a hose line, and which extending along both edges of the toroidal shape.

10. The device according to claim 9, wherein:
a) diameters of the two hollow bodies forming a ring are different, such that the elongated hollow bodies form a frustoconical wall.

11. The device according to claim 9, wherein:
a) diameters of the two hollow bodies forming a ring are the same, such that the elongated hollow bodies form a cylindrical wall.

12. The device according to claim 9, wherein:
a) an interior of multiple, or of all hollow bodies are connected to each other.

13. The device according to claim 9, wherein:
a) interiors of the elongated hollow bodies are connected in axial direction to hollow space of the one hollow body, and, alternately, to hollow space of the other hollow body forming a ring; and
b) the annular hollow bodies each have respective dedicated hose lines for filling their hollow spaces, and each have respective hollow spaces of the elongated bodies connected to the same.

14. The device according to claim 9, wherein:
a) the hollow bodies are formed by two plastic sheets, which are welded to each other in the region of the boundaries of the hollow bodies.

15. The device according to claim 9, wherein:
a) protrusions are arranged on one or both of the hollow bodies each forming a respective ring, which can be connected to adjacent body parts, particularly to the abdominal wall, by means of surgical threads, in order to secure the device.

16. The device according to claim 15, wherein:
a) the protrusions are formed by individual tabs, or by circumferential protrusions.
